(19) United States
(12) Patent Application Publication
(10) Pub. No.: US 2004/0005572 A1
(43) Pub. Date: Jan. 8, 2004

(54) Electronically readable microarrays

(76) Inventors: S. Jeffrey Rosner, Palo Alto, CA (US); Stephen S. Laderman, Menlo Park, CA (US); Dietrich W. Vook, Menlo Park, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/189,912

(22) Filed: Jul. 5, 2002

(51) Int. Cl. C12Q 1/68; G01N 33/53; C12M 1/34

(52) U.S. Cl. 435/6; 435/7.1; 435/287.2

(57) ABSTRACT

Devices are disclosed having one or more detection sites. The devices comprise a conductive layer and a plurality of separate conductive elements. The conductive layer has a plurality of openings therethrough and is at equipotential. Each of the conductive elements is exposed within a respective opening in the conductive layer and is separated therefrom by an insulating material to provide an exposed surface of the insulating material thereby providing detection sites. The detection sites or groups thereof are electrically isolated from one another. The electrical properties of the exposed surfaces are individually electrically addressable and readable by virtue of the conductive elements and the conductive layer. The exposed surface usually has a reactant attached thereto. The reactant may be the same at each detection site or the reactant at one site may be different from a reactant at another site. In one embodiment the device has within it both sensing circuitry and circuitry for statistically interpreting data sensed by the sensing circuitry. Preferably, the above circuitry is present on the same substrate. Thus, the devices may be electrically independent and the sensing circuitry may be integrated with switching being integral to the device.

### BACKGROUND OF THE INVENTION

This invention relates to any field where it is desirable to create arrays of chemical sensors with differing properties, particularly where these measurements represent information that can be used to benefit in a combinatorial fashion. Additionally, this invention relates to any physical phenomena that can be induced to provide a current or a change of electrical properties of a suitably functionalized surface. More specifically, this approach has seen current application in the field of bioscience in which arrays of oligonucleotide probes, fabricated or deposited on a surface, are used to identify DNA sequences. The present invention has a wide range of application for synthesis of arrays for conducting cell study, for diagnosing disease, identifying gene expression, monitoring drug response, determination of viral load, identifying genetic polymorphisms, and the like.

Significant morbidity and mortality are associated with infectious diseases and genetically inherited disorders. More rapid and accurate diagnostic methods are required for better monitoring and treatment of these conditions. Molecular methods using DNA probes, nucleic acid hybridization and in vitro amplification techniques are promising methods offering advantages to conventional methods used for patient diagnoses. Nucleic acid hybridization has been employed for investigating the identity and establishing the presence of nucleic acids.

Direct detection of labeled target hybridized to surface-bound probes is particularly advantageous if the surface contains a mosaic of different probes that are individually localized to discrete, known areas of the surface. Such ordered arrays containing a large number of oligonucleotide probes have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations.

In the case of commercially available DNA microarrays, the goal of array fabrication is to produce a matrix of the order of 10,000 probe features or more in an area several to tens of millimeters on a side. Each feature contains an oligonucleotide probe with a length typically in the 10 to 40 base pair length. Many methods have been put forth for fabricating such arrays. In one approach the oligonucleotide probes are spotted on a suitable surface to produce an array. In another approach, arrays are fabricated in situ, adding one base pair at a time to a primer site. Affymetrix, for example, uses photolithography to uncover sites, which are then exposed and reacted with one of the four base pair phosphoramidites. Another in situ method employs inkjet printing technology to dispense the appropriate phosphoramidite onto the individual probe sites. For example, see U.S. Pat. No. 5,700,637 and PCT WO 95/25116.

Another method involves electrochemically patterning a surface. An electrolyte overlying the surface and an array of electrodes adjacent to the surface and in contact with the electrolyte is provided. The potential of one or more electrodes of the array is altered so as to deposit or remove or chemically modify a substance on the surface adjacent the electrode. Several such treatments may be performed in sequence using different electrodes of the array. The method may be used for step-wise chemical synthesis of, for example, oligonucleotides tethered to the surface.

In a similar approach a self-addressable, self-assembling microelectronic device is used to carry out and control multi-step and multiplex molecular biological reactions, such as biopolymer synthesis, nucleic acid hybridization, antibody-antigen reaction, and diagnostics, in microscopic formats. The device electronically can control the transport and attachment of specific binding entities and other reactants to specific micro-locations.

Array plates have been discussed where a glass support surface is coated with a positive or negative photoresist substance and then exposed to light and developed to create a patterned region of a first exposed surface and a photoresist coated surface on the support. The first exposed surface is reacted with a fluoroalkylsilane to form a stable fluoroalkylsiloxane hydrophobic matrix on the first exposed surface. The photoresist coat on the surface is removed so as to form a second exposed surface, which is reacted with a hydroxy- or aminoalkylsilane so as to convert the second exposed surface to a derivatized hydrophilic binding site region and thus form the array plate.

In all the previous approaches discussed above, the arrays are created in a manner that requires an independent instrument for reading. This is a significant disadvantage of these prior methods. Another disadvantage of the arrays discussed above results from their reliance on fluorescence as the primary detection means. Such detection requires the use of proprietary dyes and difficult preparative chemistry. Furthermore, technology for reading the arrays is complex because it often requires sophisticated optics and precision mechanical motion. The aforementioned difficulties limit the applicability of the array technology and result in relatively high cost associated with array technology.

In another approach a biological electrode array is used. Each electrode in the array is coupled to a respective sample-and-hold circuit. The electrodes and sample-and-hold circuits are integral and form an array within a single semiconductor chip, such that each sample-and-hold circuit may be loaded with a predefined voltage provided by a single, time-shared digital-to-analog converter. All of the sample-and-hold may be accessed through a multiplexer that may scan through some or all of the electrode locations. Each sample-and-hold circuit may comprise a capacitor and one or more transistor switches, which, when closed, provide electrical communication between the capacitor and a source line formed in the matrix.

In another approach (Eichen, et al., WO 99/57550) an assay set is employed for detection of a target in a sample. The assay set comprises at least two spaced apart electrodes comprising a recognition moiety capable of binding to the target. The recognition moiety is attached to at least one of the electrodes or the substrate therein between. If the recognition moiety binds to the target, a conductive bridge can be formed between the electrodes based on the complex between the recognition moiety and the target. The conductive bridge is formed by using nucleation-center-forming entities attached to the complexes or to the target from which a conductive substance is substantially grown. Alternatively, the conducting bridge forms a conductive polymer between the electrodes.

### SUMMARY OF THE INVENTION

One embodiment of the present invention is a device comprising a conductive layer and a plurality of separate conductive elements. The conductive layer has a plurality of openings therethrough and is at equipotential. Each of the conductive elements is exposed within a respective opening in the conductive layer and is separated therefrom by an insulating material to provide an exposed surface of the insulating material. This combination of a conductive element exposed within a respective opening in the conductive layer and separated therefrom by an insulating material is called a “detection site”. The detection sites or groups thereof are electrically isolated from one another. The electrical properties of the exposed surfaces are individually electrically addressable and readable by virtue of the conductive elements and the conductive layer. The exposed surface usually has a reactant attached thereto. The reactant may be the same at each detection site or the reactant at one site may be different from a reactant at another site. Accordingly, the present device has multiplexing capabilities. The device may comprise a substrate that supports, and in which are contained, the conductive elements.

Another embodiment of the present invention is a device comprising a conductive layer having a plurality of openings therethrough and a plurality of separate conductive elements disposed adjacent to a respective opening. The conductive layer has a plurality of openings therethrough. The conductive layer may be supported by the substrate and is at equipotential. Each of the plurality of separate conductive elements is exposed within a respective opening in the conductive layer and separated therefrom by an insulating material to provide an exposed surface of the insulating material thereby forming a plurality of detection sites. The detection sites or groups thereof are electrically isolated from one another by virtue of the conductive layer and the conductive elements. The exposed surfaces are individually electrically addressable and readable also by virtue of the conductive layer and the separate conductive elements.

Another embodiment of the present invention is a device comprising a conductive layer having a plurality of openings therethrough and a plurality of separate conductive elements disposed adjacent to a respective opening. The conductive layer has a plurality of openings therethrough. The conductive layer may be supported by the substrate and is at equipotential. Each of the plurality of separate conductive elements is exposed within a respective opening in the conductive layer and separated therefrom by an insulating material to provide an exposed surface of the insulating material thereby forming a plurality of detection sites. The detection sites or groups thereof are electrically isolated from one another by virtue of the conductive layer and the conductive elements. The exposed surfaces are individually electrically addressable and readable also by virtue of the conductive layer and the separate conductive elements. The detection sites or groups thereof are adapted to connect sequentially, individually or in groups, to a predetermined sense circuit or predetermined sets thereof. The sense circuit is adapted to render for each detection site a determination of the electrical activity adjacent a conductive element of the detection site. Each sense circuit, or a group thereof, has associated circuitry to assemble a statistical description for a group of detection sites. In one embodiment the device has within it both sensing circuitry and circuitry for statistically interpreting data sensed by the sensing circuitry. Preferably, the above circuitry is present on the same substrate. Thus, the devices may be electrically independent and the sensing circuitry may be integrated with switching being integral to the device.

Another embodiment of the present invention is a method for assessing the status of detection sites on a substrate. In the method data is acquired electronically from multiple detection sites on a single substrate. A statistical description is then assembled for a group of detection sites by means of sense circuitry on the substrate.

### BRIEF DESCRIPTION OF THE DRAWINGS

FIG. 1 is a schematic diagram depicting an array comprising a device in accordance with the present invention.

FIG. 2 is a cross-sectional view of one embodiment of a device in accordance with the present invention depicting a particular CMOS realization showing four detection sites arranged symmetrically with respect to their readout circuitry.

FIG. 3 is a simplified electronic circuit diagram indicating the usage of a CMOS transistor as a switch for the detection sites depicted in FIG. 2 in accordance with the present invention.

FIG. 4 is a top view of the layout shown in FIG. 1 in cross-section.

FIG. 5 is a cross-sectional view of an alternative embodiment in accordance with the present invention depicting a CMOS realization wherein the surface is rendered as a planar structure.

FIG. 6 is a top view of the layout shown in FIG. 5 in cross-section.

### DETAILED DESCRIPTION OF THE INVENTION

In its broadest application the present invention is directed to devices and methods for carrying out reactions involving reactants such as, by way of illustration and not limitation, hybridization reactions involving biopolymers. The device comprises an array of sensor elements or detection sites, which are chemically and electronically isolated, in conjunction with a top layer that is at equipotential, e.g., ground. The array is generally a plurality of the sensor elements arranged in a predetermined pattern such as columns and rows and the like.

In the devices of the present invention, sensor element activity is only engaged with respect to a small exposed piece of underlying conductive element such as metal, which is otherwise isolated. The top layer functions as a guard plane that is only interrupted, for example, perforated, at active sites with all the sensor elements exposed only at these interruptions. Accordingly, sensor elements are electrically isolated from one another. The sensor elements are actuated or interrogated electronically. Measurements may be made digitally directly from the device as opposed to external measurements that are required in the devices of the prior art. The devices and methods of the invention, utilizing a conductive layer at equipotential and isolated separate sensor elements or detection sites, avoid the situation wherein exposed glass potentially becomes a sensor effectively shorting together all such sensors in the array, which is potentially the case with the devices of, for example, Eichen, et al., supra.

The devices of the invention utilize a conduction path, which can be made extremely short, across an exposed surface of an insulating material that lies between a top layer at equipotential and a sensor element. The conduction path is determined by the thickness of the layers rather than by lithography as in prior art devices. In one embodiment the devices comprise an active matrix array of read elements analogous to a read-only memory, or ROM, each associated with a specific sensor element. Accordingly, the device can be the entire instrument itself. The conductivity associated with each sensor element may be modified by a target event. The technology surrounding sensor materials currently used in “electronic nose” technology or other current polymer sensors may be applied to the devices of the present invention. Signals from various types of sensing schemes may be read on the present device. The basic concept of this invention is a micro-electronic device with specially designed addressable and readable microscopic locations. The integration with commercially available CMOS circuitry that is enabled by this device can effectively contain a low-cost sensor integrated with its own instrument in a monolithic array. In this way the present device effectively reads itself.

The separate conductive elements are individually electrically addressable and readable. In use a plurality of detection sites of the reaction device are brought into proximity with a reaction medium. The reaction medium comprises reagents for carrying out the reactions. The metal elements are selectively electrically addressed by selectively activating the electrical leads. An electrical response is selectively read from individual metal elements. The reactions result in the ability to close an electrical circuit between the metal elements and the metal layer in each of the openings in which a hybridization reaction occurs or the ability to selectively drive an electrochemical reaction based on the chemical or biological state at the metal element.

The present device may be employed in the determination of a targeted event. Generally, the targeted event is a targeted chemical reaction, which may be, for example, diagnostic reactions such as, e.g., a DNA hybridization reaction, a peptide/receptor target capture, and the like, an electrochemical deposition and so forth. The devices and methods of this invention allow important reactions such as diagnostic reactions to be carried out under complete electronic control. The devices may be employed in assay methods for the qualitative and/or quantitative determination of one or a plurality of substances of interest sometimes referred to in the art as analytes or target substances. The analyte or target substances may be those substances identified below under the discussion of reactants. The present devices may be used in most known applications in which arrays are utilized, provided a suitable chemistry is available to provide conductivity modification or electrochemical current to mark targeted events at detection sites. For example, the devices may be employed to determine one or a plurality of target nucleic acids using attached polynucleotides that are directed to the same target nucleic acid or each directed to one or more of a plurality of target nucleic acids. The instant devices also may be used in sequencing of target polynucleotides or in determining single nucleotide polymorphisms. These devices may also be used to selectively drive and/or monitor electrochemical reactions. All that is required to use such a device is the existence of a useful reaction that can modify the conductivity of a glass surface or produce an electrochemical current at an electrode.

In one embodiment a device for carrying out reactions comprises a conductive layer, optionally supported by a substrate, and a plurality of separate conductive or sensor elements optionally within the substrate. The conductive layer and the plurality of separate conductive elements are isolated from each other by an insulating material. The basic feature of the insulating material is that it provides electrical isolation of the conductive layer from selected members of the plurality of separate conductive elements and electrical isolation of the members of the plurality of separate conductive elements from one another. In this way, independent electrical circuits between the portions of the conductive layer and selected members of the separate conductive elements may be created as a result of a reaction adjacent a separate conductive element. Accordingly, the insulating material need only limit the ability of a circuit being closed between a portion of the conductive layer and a selected member of the separate conductive elements. In general, the insulating material is comprised of a material that is relatively nonconductive with respect to the conducting layer and the conducting elements. Such materials are usually insulators such as, by way of illustration and not limitation, silicon dioxide, certain metal oxides, certain plastics, ceramics, silicon nitride, alumina, nitrides and oxides of silicon and many metals and the like. The supporting substrate may be an inert substance, i.e., a substance that does not exhibit substantial reactivity with the reactants or components of a reaction medium, for example, silicon, glass, polymer, sapphire, many ceramics containing oxides or nitrides of metals and the like.

The conductive layer and the conductive elements are formed from a material that readily conducts electrical current such as a metal, doped semiconductor, conductive plastic and so forth. Such metals include, by way of example and not limitation, gold, platinum, nickel, copper, silver, aluminum, tin, palladium, titanium nitride, titanium-tungsten alloys, and so forth and alloys and compounds of such metals.

The conductive layer is supported by the substrate and is usually deposited upon the substrate by techniques well known in the art such as, for example, chemical vapor deposition, DC or magnetron sputtering, vacuum evaporation, plating and the like. Generally, the thickness of the conductive layer is about 100 to about 20,000 nanometers. The conductive layer is usually at equipotential, that is, the potential is the same throughout the conductive layer. In a preferred embodiment the potential of the conductive layer is at ground. The potential of the conductive layer may be in the range of about −100 to about +100 Volts, usually, in the range of about −5 to about +5 Volts

The conductive layer has a plurality of openings or holes therethrough. These holes are typically on the order of microns, i.e., about 0.1 to about 500 microns, usually, about 0.2 to about 10 microns, more usually, about 1 to about 5 microns, in diameter. The holes may be formed by microlithographic or other techniques well-known in the art of IC design such as electron beam lithography, ion beam lithography, or nano-imprinting. Micromachining techniques or ablation may also be employed to create the openings in the conductive layer. While microscopic detection sites are desirable for some applications such as high density arrays, larger addressable sites (e.g., larger than 2 mm) may be employed for some purposes. Accordingly, the size of an opening can be of any size, usually in the range from about 0.2 microns to about 2 millimeters or more. A device can be designed to have as few as two openings or as many as hundreds of thousands of openings. The openings may be of any shape such as square, rectangular, circular, oval, serpentine and so forth. Preferably, the openings are square or rectangular for maximizing their area or linear or serpentine for maximizing the length of the edge of the conductive layer opening.

Each of the separate conductive elements is exposed within a respective opening in the conductive layer and is separated therefrom by the insulating material to provide an exposed surface of the insulating material. By the term “exposed within” is meant that each of the conductive elements is accessible within the respective opening for carrying out a target chemical reaction. Each of the separate conductive elements is electrically or conductively isolated from the conductive layer and from each other by the insulating material. Usually, the conductive elements are formed within the substrate by techniques well known in the art such as, for example, methods similar to the aforementioned forming of holes and additionally including etched metal, damascene metal formed by inlay and polishing, doped single crystal or polycrystalline regions and the like. Effectively, there is a plurality of openings in the substrate, on which the conductive layer is supported, corresponding to the number of openings in the conductive layer. These openings may be formed in a manner similar to that discussed above for forming the openings in the conductive layer. Likewise, the shape and dimensions of the openings correspond to that of the openings in the conductive layer

The arrangement of the openings in the conductive layer with respect to the conductive elements creates a plurality of detection sites, whose edges are the edges of the openings and whose conductive elements are separated from the conductive layer by an exposed surface of insulating material between the edges of a respective opening in the conductive layer and a corresponding edge of the conductive element. The width of the exposed surface generally corresponds to the thickness of the layer of insulating material between the conductive layer and the conductive elements.

The exposed surface of insulating material and/or the conducting element in each of the detection sites has attached thereto one or more reactants. The reactant at each detection site may be the same or the reactant at one detection site may be different from a reactant at another detection site. The reactant is a biological or non-biological substance that is environmentally sensitive, i.e., sensitive to the environment adjacent to the attached reactant. The reactant may undergo a chemical or physical reaction with one or more components of the environment or the reactant may be electrochemically sensitive within the environment. The reactant may be a member of a binding pair such as protein/receptor pair, a set of matched single-stranded DNA or other similar sets. The biological substances include biopolymers, immunoreactants such as antigens and antibodies, receptors such as avidin, thyroxine binding globulin, thyroxine binding prealbumin, transcortin etc., and the like. The non-biological substances include electrochemically responsive compounds (compounds that respond to an electrochemical stimulus) including, for example, electrochemically active electrodes as used in medical assays or clinical laboratories, and the like, photochemically responsive compounds (compounds that respond to a photochemical stimulus) including, for example, dyes, photosensitive catalysts and the like.

The exposed surface either has a functional group for attachment or must be treated or modified by chemical techniques to provide such a functional group. Representative groups include, by way of illustration and not limitation, amino, especially primary amino, hydroxyl, thiol, sulfonic acid, phosphorous and phosphoric acid, particularly in the form of acid halides, especially chloride and bromide, and carboxyl, and the like. A procedure for creating the attachment chemistry is sometimes referred to as “priming” the surface. To this end, the exposed surface is modified so as to prepare the surface for attachment of the reactant. The reactant may be attached directly to the exposed surface or it may be synthesized on the surface. In the former approach the reactant comprises a functional group for attachment. In the latter approach the reactant is formed in situ such as, for example, the formation of biopolymers by employing monomeric building blocks such as nucleotide triphosphates.

An important consideration in treating the exposed surface to generate a necessary modification is that the adjacent conductive layer and conductive element should not be prevented from functioning. The exposed surface may be modified with groups or coupling agents to covalently link the biopolymer. The reactive functional groups may be conveniently attached to the exposed surface through a hydrocarbyl radical such as an alkylene or phenylene divalent radical. Such hydrocarbyl groups may contain up to 10 carbon atoms.

One preferred procedure for the derivatization of the exposed surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with oxide and/or hydroxyl groups, which are present on or introduced on the exposed surface. APS provides primary amine groups for the subsequent covalent coupling reactions of the oligonucleotide synthesis. Such a procedure is described in EP 0 173 356 B1, the relevant portions of which are incorporated herein by reference. While this represents one of the approaches, a variety of other attachment reactions are possible for both the covalent and non-covalent attachment as mentioned above.

The biopolymers may be, for example, polynucleotides, poly(amino acids), polysaccharides, mucopolysaccharides, nucleic acids, and combinations thereof. Such combinations include components of bacteria, viruses, chromosomes, genes, mitochondria, nuclei, cell membranes and the like. A polynucleotide or nucleic acid is a compound or composition that is a polymeric nucleotide or nucleic acid polymer, which may include modified nucleotides.

The combination of the openings in the conductive layer and corresponding conductive elements results in detection sites or sensor elements on the device. The number of detection sites as well as the size of each site are governed by number of factors such as the nature, complexity, and amount of the analyte, the desired level of sensitivity, cost, chip yield, and the like. The number of sites may be from 1 to about 16 million, usually about 100 to about 10,000 and the size of each site is discussed above in the discussion regarding the openings in the conductive layer. Generally, sites with greater area are more sensitive for reactions involving direct electrochemical voltammetric sensing. Sites with more perimeter length of exposed insulating surface separating the edge of the conductive elements from the edge of the openings in the conductive layer are more sensitive for measurements of modification of surface properties, as in specific binding techniques using a conductivity tagged analyte.

For the purpose of utilization in a large variety of applications, these detection sites can be arranged in groupings that we will call ‘features’ both logically and physically. The physical grouping or feature can be arranged for the purpose of allowing each detection site within such a feature to be conveniently treated or modified at one time by chemical modification in a single operation or sequence of operations. This creates a feature with identical chemical activity at each site. These features can contain from one to millions of identical sites, most typically 10 to 1000.

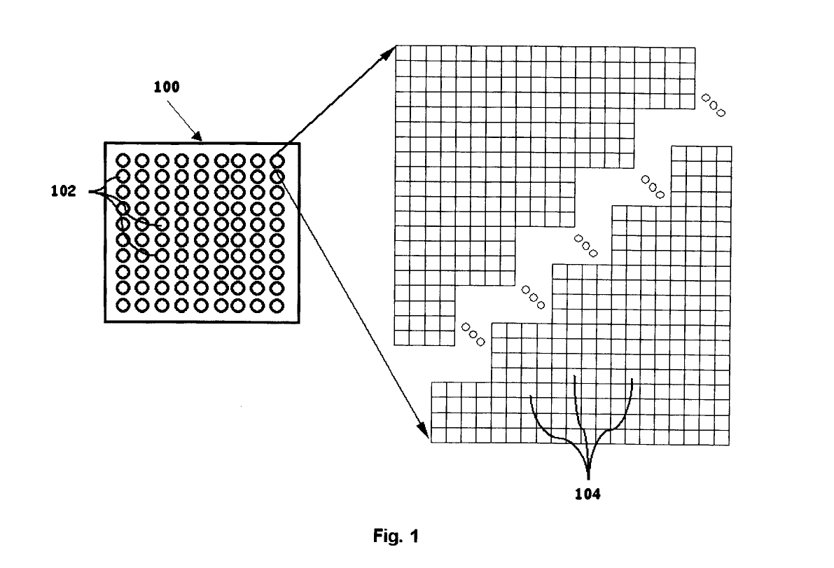

A given device may have from 1 to 1,000,000 such features but more typically 100 to 10,000. By assembling statistical reports or histograms of the responses from all the sites within a feature and comparing the result to knowledge of such a response, a quantitative measure of properties of the analyte such as concentration, reactivity, nucleotide sequence mismatches, etc. can be ascertained. Like current practice in the application of DNA micro-arrays, information from collections of these features can be used in a combinatorial fashion to determine the nature of the analyte. Unlike current practice in the present invention the aforementioned function, namely, assembling a statistical description for a group of detection sites, is accomplished by means of sense circuitry integrated into the devices of the invention. A typical arrangement of the sites and features is depicted schematically in FIG. 1 by way of illustration and not limitation.

The size of the overall device will depend on a number of factors such as the density of the detection sites, the number of detection sites per feature (determined by the dynamic range requirement) the cost requirement, area required by the instrumentation electronics, and the particular manner in which the device is used.

As mentioned above, the separate conductive elements are individually electrically addressable and readable by virtue of electronics that are onboard the present devices. Accordingly, a device in accordance with the present invention usually comprises a plurality of electrical leads coupled to one or more of the conductive elements for electrically individually addressing the separate conductive elements. The electrical leads may be formed by any technique and material typically used for electrical connections in a thin-film circuit, being patterned and/or multi-layered structures of metals, doped semiconductors, conductive organic films, and the like. Furthermore, the device can comprise an electrical lead coupled to one or more of the conductive elements for individually reading an electrical signal generated adjacent the conductive element.

In one embodiment the electrical lead for individually addressing separate conductive elements is coupled by a switch to an electrical lead for individually reading an electrical signal generated at the conductive element. In this way each switch may be activated individually and, if an electrical signal is present adjacent the conductive element, the signal may be read by an appropriate reading element or reading instrumentation either externally disposed or integrated within the device. In general, the switch is a component having more than one terminal where the conductivity between two of the terminals may be turned on or off in a controlled manner. The switches may be, for example, a T memory cell such as a transistor, e.g., an NMOS transistor, a PMOS transistor, bipolar transistor, thin film transistor or any multiterminal electronic device that exhibits gain, etc., and so forth. In this way a number of electrical leads coupled to metal elements for individually addressing and a number of electrical leads coupled to metal elements for individually reading are multiplexed, i.e., connected to a fewer number of sense circuits. In this manner, each of a plurality of sense circuits can sequentially read some fraction of the total number of detection sites. By the term “sense circuit” is meant an electronic circuit designed to measure a charge, current or electrical resistance at a detection site. The rows and columns of such an array can be addressed in a row sequential or column sequential manner. Often column addresses are called word lines and rows are bit lines, in the manner of computer memory chips. The readout can then proceed in a manner identical to computer memories. A discussion can be found in “Semiconductor Memories”, 2^{nd} edition, by Betty Price, Wiley and Sons, ©1983, reprinted 1996.

In one embodiment the sensor array is addressed in a manner similar to that of a memory with a grid of source lines connected to switches (MOSFETs) at each of the detection sites and column or word lines carry signal from the detection sites to a sensing element. The word lines short together the drains of all of the switching transistors of a given column. On a given word line there may be from just a few sense transistor drains to many thousands. The larger the number of drains on each word line, the more sensitive the sense amplifier will need to be. Each bit line connects to the gate of only one transistor switch associated with each word line.

The separate conductive elements are adapted to connect sequentially to a predetermined sense circuit or predetermined sets thereof. The sense circuit is adapted to render for each detection site a determination of the electrical activity adjacent a conductive element of said detection site. The electrical activity may be, for example, resistivity between the conductive layer and the conductive element of each of the detection sites. Alternatively, the electrical activity may be current produced at the conductive element of each of the detection sites.

In one embodiment a group of detection sites is adapted to connect sequentially to predetermined sets of sense circuits. In another embodiment a group of detection sites is adapted to connect sequentially in a predetermined manner to a single sense circuit. In yet another embodiment each of the detection sites is adapted to connect sequentially to a predetermined sense circuit. In another embodiment the device is adapted to collect statistical data and assemble a statistical description from a group of elements such as a feature. To this end the device comprises appropriate circuitry wherein a single sense circuit or a group of sense circuits may be employed for collecting data from multiple detection sites and the output is read as a single number or a reduced set of numbers that describes the distribution of results within the feature such as, for example, a histogram. This embodiment is in contrast to reading each detection site one at a time and assembling a statistical description of the collected data with external instrumentation and computation equipment. By the phrase “assemble a statistical description” is meant taking a collection of numbers, each representing the state of a detection site and reducing the collection to a substantially smaller set using common statistical processes such as, for example, a histogram, a mean and standard deviation description, and the like.

The nature of the reading element is dependent on the nature of the electrical signal or electrical activity. For example, where the electrical signal is current, an appropriate element for reading current is employed. Such elements include current sensors such as in voltammetry or when a fixed potential is applied across the element, e.g., Sense Amps, current amplifiers with threshold discriminators, differential amplifiers, and analog/digital converters, etc., and the like. Where the electrical signal is a voltage, a high impedance differential amplifier is typically used.

In one embodiment a saturating amplifier is used, so as to produce either of two distinct output states (“1” or “0”). This invention also encompasses the use of an analog/digital converter or direct output of the analog values. These analog values correspond to changes in the resistivity, or other electrical parameter, at a specific detection site.

The devices in accordance with the present invention include all internal and external circuitry for addressing and reading the aforementioned elements of the devices. Much of the external circuitry is known in the art and includes electric or electronic components such as standard solid-state microelectronic components. The devices may be interfaced with a computer for controlling the addressing and reading functions and collection and storage of data, or the data may be collected on the chip for statistical reporting of selected results

In many reactions of the type subject to the present devices, the result on each element will be a single discrimination of whether a targeted event, i.e., a targeted chemical reaction, has occurred. As discussed above, the targeted chemical reaction may be, for example, a DNA hybridization reaction, a peptide/receptor target capture, an electrochemical deposition and so forth. More generally, it is desirable to gain a proportional measurement, e.g., a numeric output proportional to concentration. In these cases, the probability of the targeted event occurrence is nearly always monotonically related to, or more desirably, directly proportional to the desired measurement variable. For this purpose, large numbers of identically functionalized elements can be logically (and physically, for convenience of functionalization) grouped in collections, which may be referred to as features. The statistically collected occurrence of the targeted event within a feature can then be determined by counting of ‘positive’ sites combined with a tabular or analytical representation of the linear or non-linear relationship between event probability and desired measurement variable. The number of elements in a feature for a given desired accuracy is then determined by counting statistics, where the uncertainty (or noise) of the measurement is proportional to the square root of the total count (i.e. the uncertainty of a count of 100 positive events is +/−10, uncertainty of 10,000 is +/−100, and so forth).

Along with the sensor elements, each feature can have “accumulator” circuitry that accumulates a count of positive events within a feature, resulting in the ability to directly read the result of a large number of measurements in a single number. Additionally, the tabular knowledge of such an assessment can also be built, by hardware, firmware, or software, into the device to allow a calibrated, qualified measurement to directly result from the present device without external electronics.

In the present invention the determination of electrical activity determines a chemical or biological state of the detection site. The ability to generate an electrical signal at the exposed surface generally results from a response of a reactant with its environment. As mentioned above, the response may be a reaction with a component of a medium, an electrochemical reaction, and the like. The reaction of a reactant with a component in its environment may involve the covalent or non-covalent binding of the reactant with the component. For example, for a reactant that is a polynucleotide, the reaction may be a hybridization event adjacent the separate conductive element. The hybridization event may arise, for example, from the hybridization of a target polynucleotide sequence to the polynucleotide attached to the exposed surface adjacent the separate conductive element. The target nucleotide sequence is a sequence of nucleotides to be identified, usually existing within a portion or all of a polynucleotide, usually a polynucleotide analyte. The identity of the target nucleotide sequence generally is known to an extent sufficient to allow preparation of various sequences hybridizable with the target nucleotide sequence and of oligonucleotides, such as probes and primers, and other molecules necessary for conducting methods in accordance with the present invention. The target sequence usually contains from about 30 to 5,000 or more nucleotides, preferably 50 to 1,000 nucleotides. The target nucleotide sequence is generally a fraction of a larger molecule or it may be substantially the entire molecule such as a polynucleotide as described above.

The hybridization or binding of the target sequence to the attached polynucleotide results from the ability of two nucleotide sequences to hybridize with each other. The ability to hybridize is based on the degree of complementarity of the target sequence and the attached polynucleotide, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of cosolvents, lowering the salt concentration, adding a surfactant, and the like.

In one embodiment the target sequence comprises, or is adapted to comprise, a material for providing an electrical circuit between a respective separate conductive element and the conductive layer. For example, the target sequence may comprise a metal such as gold, silver, copper, nickel, iron, a photosensitive material such as TiO₂, or an electrochemical plating catalyst and the like and combinations thereof as well as metal aggregates, complexes, clusters and the like such as colloids and so forth, which acts to close the aforementioned electrical circuit.

For purposes of illustration and not limitation, in one embodiment the target sequence may be treated to introduce gold into the target sequence molecule. The introduction of gold may be carried out prior to or subsequent to the hybridization event. In either circumstance a group may be introduced into the target sequence prior to provide for introducing gold into the target sequence. The group may be, for example, a small organic molecule of molecular weight of about 50 to about 2000, usually, about 100 to about 500, such as, e.g., biotin, fluorescein, dinitrophenol, and the like. Either prior to or subsequent to the hybridization event, gold may be introduced into the target sequence by using a binding partner for the small organic molecule, such as, streptavidin, antibody to fluorescein, antibody to dinitrophenol, etc., attached to gold. See, for example, Nanogold® from Nanoprobes, Inc., Stony Brook, N.Y. After introduction of the gold, a plating process may be employed to plate additional gold at the site where the target sequence is hybridized thereby closing the electrical circuit and permitting an electrical signal to be read. The synthesis and use of gold attached to immunoreactants is discussed in more detail in Velev, et al., Langmuir, The ACS Journal of Surfaces and Colloids (1999) 15(11):3693-3698. The above reference utilizes colloidal gold and its enhancement by silver nucleation. Other references relating to the use of gold include U.S. Pat. No. 5,284,748 and WO 99/57550, supra.

In another approach, silver metal is vectorially deposited along the target sequence hybridized to the attached polynucleotide. The process is based on the selective localization of silver ions along DNA through silver ion/sodium ion ion-exchange and formation of complexes between the silver and the DNA bases. The silver ion/sodium ion-exchange process is monitored by following the almost instantaneous quenching of the fluorescence signal of labeled DNA. Silver ion exchanged DNA is then reduced to form nanometer sized metallic silver aggregates bound to the DNA skeleton. These silver aggregates are subsequently further developed using an acidic solution of hydroquinone and silver ions under low light conditions. A discussion of this technology may be found in Nature (1998) 391:775-778 (Braun, et al.).

The devices of the present invention may be fabricated according to procedures and principles well-known to those skilled in the art of digital and IC design. The devices may be manufactured from silicon wafers, glass or polymer thin films with active electronics built in single crystal silicon, polysilicon, polymer semiconductors or glass with metal wiring. Reference books that are exemplary of those directed to the above include VLSI Technology by S. M. Sze (1988) ISBN 0-07-062735-5 and Basic VLSI Design by Pucknell and Eshraghian (1988) ISBN 0 7248 0105 7. For relatively high density arrays, such techniques include, by way of example and not limitation, fine-line IC processes known in the art such as CMOS processes, e.g., CMOS-14, bipolar processes, MOS processes, NMOS processes, ECL, flat panel thin-film transistors, and so forth. Standard design rules may apply. The design rules are determined by the tolerances of many of the fabrication equipment, but are not critical to this invention. This invention is scalable to smaller sizes, as the manufacturing technology becomes capable or larger sizes as the requirements of the application dictate. This scalability is largely enabled by the isolation of the detection sites that is attained by the continuous conductive layer of the present devices.

Embodiments of devices in accordance with the present invention are depicted in FIGS. 1-4. FIG. 1 depicts an example of a DNA microarray, by way of illustration and not limitation, to indicate the organization of the detection sites and features within the device. In general, a large array of detection sites make up a single feature in a sensor array, i.e., an array in accordance with the present invention. One such sensor array, for example, is a DNA microarray, which may have many different pieces of DNA spotted onto a solid surface. Each spot or feature of DNA on the microarray can probe for its complement in a sample to be analyzed. A given sensor array will have a number of features determined by the application; current applications for DNA arrays require from 100 to 10,000 features. Within each feature many detection sites will be grouped, generally about 100 and about 10,000, as required by the particular application to which the present device is applied and/or limited by the technological or actual embodiment of the device. The detection sites are as described above and can be edge-sensing or area sensing. The term “edge-sensing” means that the conductivity across the surface of the insulating layer resulting from a targeted chemical reaction is measured at each detection site. The term “area-sensing” means that the current produced at an electrochemical reaction on the surface of the conducting element is measured.

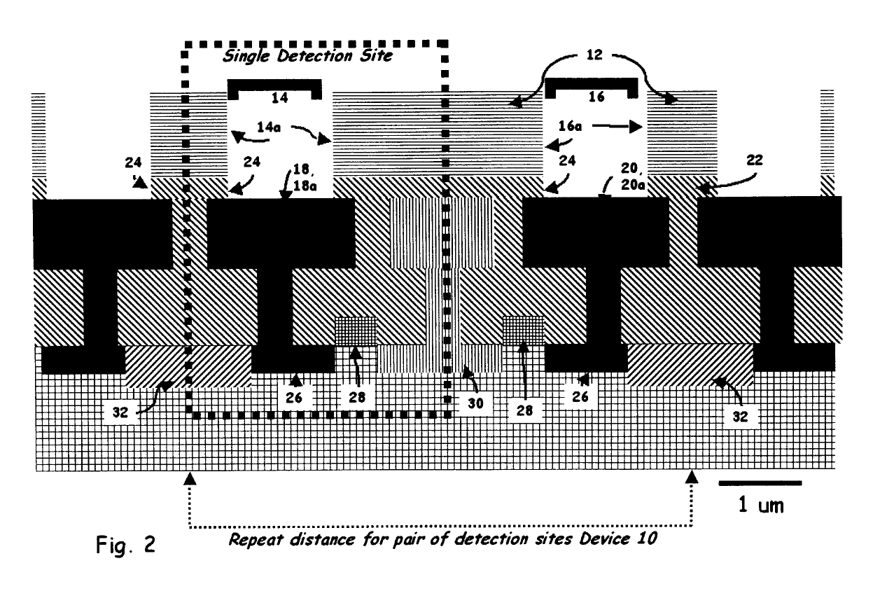

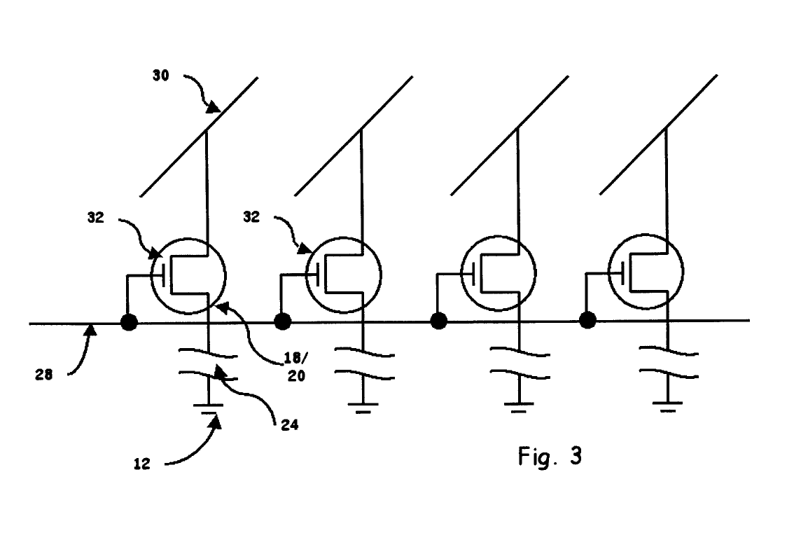

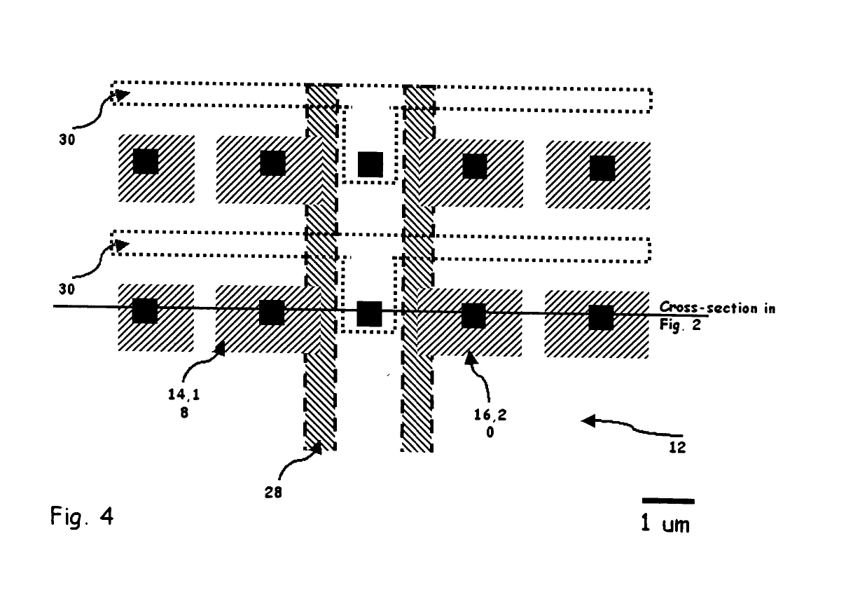

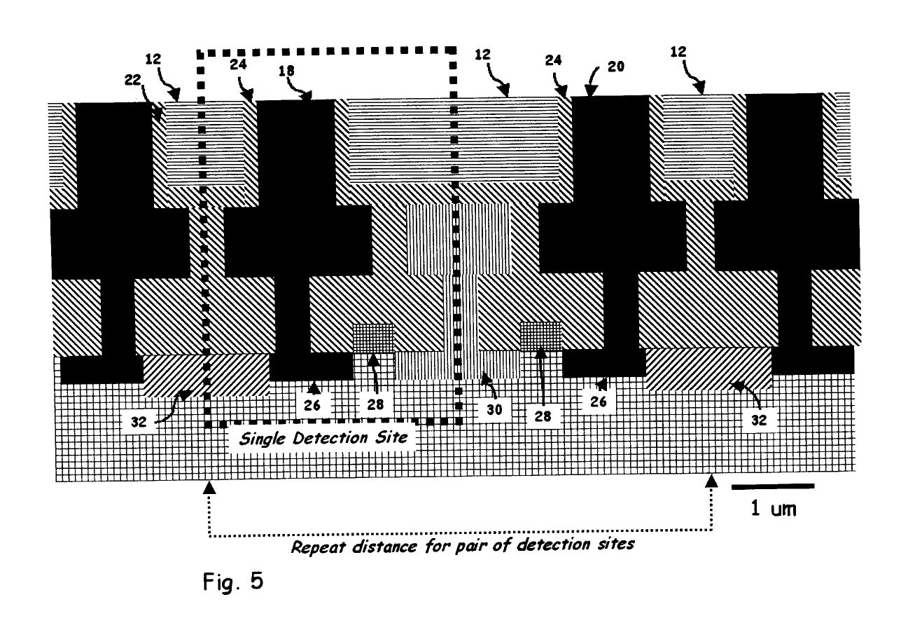

FIGS. 2 and 5 depict two examples, by way of illustration and not limitation, of specific structures or embodiments of the present invention. FIGS. 2-6 show different aspects of various embodiments of an array of detection sites in accordance with the present invention complete with a single transistor readout circuit per site. FIGS. 2 and 4 show a cross-sectional and layout view respectively of a particular CMOS realization showing four detection sites arranged symmetrically with respect to their readout circuitry in accordance with the present invention. FIG. 3 further shows the electronic circuit diagram representing such a realization. FIG. 5 shows an alternative embodiment of a device of the invention that is fabricated to yield an entirely planar structure. The device of FIG. 5 is a fully planar embodiment prepared using damascene technology.

Readout of any of these structures can be accomplished in the manner of DRAM computer memories as previously described. This embodiment may have significant advantage in accessibility of a fluid to the active surfaces as design rules continue to allow further device miniaturization. This structure can be fabricated by a technique pioneered by IBM called damascene, after the ancient Middle Eastern art of inlay. In this process the conductive element that is connected to the underlying metal is prefabricated to its final shape. Then, the insulating layer is deposited in a conformal manner, e.g., using chemical vapor deposition or spin coating as is typical in the thin film circuit industry, to coat the conductive element. Finally, the conducting layer is deposited to a substantial thickness over the whole structure. Next, the structure is polished using chemical-mechanical polishing, as is the current art for planarization in the semiconductor processing technology. The polishing proceeds until the conducting element is intersected, revealing the structure as shown. Further discussion of the aforementioned process may be found in Multilevel Interconnect Technology III by Mart Graef (Editor), Divyesh N. Patel (Editor), Society of Photo-optical Instrumentation Engineers; ISBN: 0819434809.

FIG. 1 depicts microarray 100 comprising a plurality of features 102, which are spots of DNA. Each feature 102 is comprised of a plurality of detection sites 104.

FIG. 2 depicts in cross-section a portion of a device 10 with conductive layer 12 having openings 14 and 16 therein. The rectangular area designated by broken lines represents a single detection site with its accompanying electronics. The repeat distance for openings 14 and 16 in device 10 is 6 microns. Of course, the repeat distance is dependent on the number of detection sites on the device and the dimensions of the entire device. Standard 0.5 micron CMOS design rules apply with respect to device 10. Below openings 14 and 16 are conductive elements 18 and 20, respectively, which are isolated from one another and from the conductive layer by insulating material 22, which has openings patterned simultaneously with and self-aligned to openings 14 and 16 in conductive layer 12. The combination of walls 14a and 16a of openings 14 and 16, respectively, in conductive layer 12, the surfaces 24 of insulating material 22 together with top surfaces 18a and 20a of conductive elements 18 and 20, respectively, form detection sites at openings 14 and 16. As can be seen, exposed surfaces 24 of insulating material 22 are found at the bottom of the detection sites. Biopolymers, for example, can be attached to the exposed surfaces, which act as detection sites for hybridization events that occur involving the attached biopolymers and modify the conductivity of the exposed surfaces 24. Alternately, reagents for promoting selective electrochemical reactions can be attached to surfaces 18a and 20a, acting as detection sites for the presence of targeted compounds in the local environment to which the reagents are sensitized.

Referring to FIGS. 2 and 3, device 10 further comprises source electrical leads 26 that connect to the detection sites as discussed more fully below. Device 10 also comprises bit lines 28 and word line or common drain 30. Bit lines 28 provide electrical leads to conductive elements 18 and 20 as well as connection to common drain 30. Field oxide (FOX) is designated 32 in FIG. 1. Device 10 also comprises transistors 32 as shown in FIG. 2. FIG. 3 shows the electrical schematic of a ‘row’ of detection sites indicating how the bit line 28 would be shared among the detection sites within a row. Similarly, the detection sites could be arranged in ‘columns’ where the word lines 30 would be shared among detection sites. In a typical embodiment, each of the word lines 30 would be connected to the input of a sense amplifier, with at least one sense amplifier for each column, and then a single bit line 28 would be enabled, connecting an entire row of detection sites to their respective sense amplifiers.

In the schematic, the connection between the conducting layer 12 and the conductive elements 18 and 20 is indicated as the insulating surface 24; this is for the embodiment where changes in the conductivity of the surface 24 are being measured. In the embodiment where an electrochemical current is being sensed at the conductive element 18/20, this element represents the electrolytic conductivity of the environment adjacent to the detection site.

FIG. 4 depicts a layout of a CMOS circuit shown diagrammatically in cross-section in FIG. 2. This can be directly interpreted as described above. Note that the openings 18 and 20 correspond directly with the conductive elements 14 and 16 respectively. In actual realization, these conductive elements are likely to extend below the insulating layer 22 (not shown in this view) for reasons of manufacturing and alignment of the various layers to each other. Also important to realize is that the conducting layer covers the entire surface of the active region of such a device with the exception of openings such as 18 and 20.

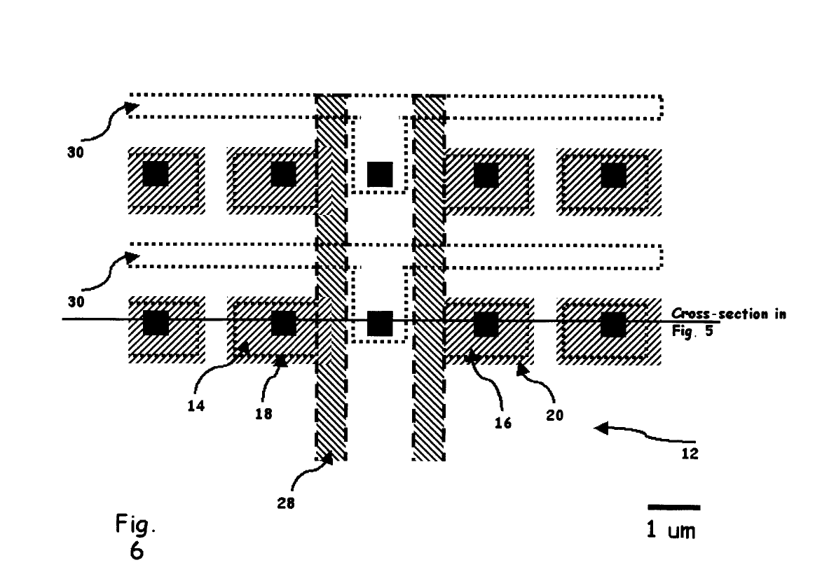

FIG. 5 shows an alternative embodiment as described previously regarding the completely planar realization of such a device using ‘damascene’ technology. This differs only in topography from the description of the functions previously described in the surface 24 of the insulating layer 22 is now rendered coplanar with the surfaces of the conductive elements 18a and 20a, as well as the conducting layer 12. FIG. 6 shows a CMOS layout corresponding to a cross-section of FIG. 5. Separate features are shown now for the conductive elements 18 and 20 since this surface area is now smaller than the openings 14 and 16 in the conductive layer 12 by an amount equal to the width of the exposed surface of the insulating layer 24.

One possible embodiment of the present invention involves a method for carrying out hybridization reactions involving biopolymers such as polynucleotides. A plurality of detection sites of a reaction device is brought into proximity with a reaction medium a plurality of detection sites of a reaction device. The device may be, for example, a device similar to that depicted in FIG. 1 comprising 1000 detection sites in a 100×100 micron square feature. Such a device is consistent with current practice in the biotechnology field. All of the detection sites within each feature have been previously identically treated in a manner consistent with current DNA arrays to attach a single-stranded probe DNA type to the exposed surface of the insulating layer. Accordingly, each additional feature within the device can be similarly treated to attach the same or distinctly different DNA sequences as the application requires. The reaction medium comprises reagents for carrying out the hybridization reactions. One of the reagents is the polynucleotide analyte. For example, in one approach, cell matter is lysed, to release its DNA as fragments, which are then separated out by electrophoresis or other means, and then treated to introduce gold into the DNA. As mentioned above, streptavidin-gold may be employed. The medium is usually an aqueous buffered medium at a moderate pH, generally that which provides optimum assay sensitivity. Such media as known to those skilled in the art and will not be discussed further here.

The reaction medium is contacted with the detection sites of the device of FIG. 1 and the medium is incubated for a time and at a temperature that optimizes the hybridization of polynucleotide analyte to the oligonucleotides present in the detection sites. Moderate temperatures are normally employed. For polynucleotide hybridization reactions, relatively low temperatures of from about 50° C. to about 80° C. are employed for the hybridization steps, while denaturation is carried out at a temperature of from about 80° C. to about 100° C. The period of incubation should be sufficient to permit the hybridization reactions to occur. The period for incubation generally ranges from about 1 second to about 24 hours or more, usually 30 seconds to 6 hours, more usually from about 2 minutes to 1 hour.

The amount of the polynucleotide analyte present may be from about 1 to about 10¹⁰, usually from about 10³ to about 10⁸ molecules, preferably at least about 10⁻²¹M or greater in the medium and may be about 10⁻¹ to about 10⁻¹⁹M, more usually about 10⁻⁵ to about 10⁻¹⁰M. It is within the scope of the present invention to amplify the amount of polynucleotide analyte prior to conducting the method in accordance with the present invention. The polynucleotide analyte may be amplified by well-known techniques such as, for example, PCR, LCR, NASBA, and so forth.

After the polynucleotide analyte is allowed to hybridize to the array of oligonucleotide probes on the present device in a manner consistent with current convention, the array is then washed. An electroplating process is carried out to plate out additional gold at the detection sites. To this end the gold plating process is carried to the extent necessary to ensure that the resulting gold particle is sufficiently large to span the exposed insulating surface providing increased conductivity between the conducting element and the conducting layer. In this manner, even a single hybridization event within a site can be detected. Any specific or non-specific binding process of biological molecules can similarly be instrumented, provided a suitable means for modifying surface conductivity can be provided.

Alternatively, the scheme described above could substitute a reactive structure designed to promote an electrochemical reaction in place of the DNA probe molecule to allow the electrochemical reactions to be selectively driven as each detection site is activated. By way of example and not limitation, this embodiment may be a plating reaction seeded by the Nanogold described previously, a reaction driven by the absorption of a photon from an external stimulus causing the transport of electrical current from an electrolyte to the appropriately functionalized site, and so forth.

In one readout scheme, the conductive layer edges of the detection sites of present device are maintained at ground. That is, the entire conductive layer 12 is maintained at ground potential. The conductive elements of the detection sites are selectively electrically addressed and an electrical response is selectively read therefrom. Referring to FIGS. 2-4, one word line is provided with a nominal source current, which is determined by the current compliance of the sensor type. The bit lines are sequentially brought up well above Vt for the transistor. If a detection site provided a current by virtue of a hybridization reaction and subsequent gold plating, or by virtue of an electrochemical reaction being driven to provide a measurable ion current, then the transistor would turn on and the voltage at the sense amp/word line is the source current divided by the sum of the transistor on resistance and the detection site resistance. The sense amp reporting function can include on-chip circuitry that collects the statistics (percentage of sites within the feature that exhibit conductivity or electrochemical current above a certain threshold) and provides a numeric output similar to a known bio-chip reader. This numeric output would be a digital output that could represent any technologically important summary of the statistics within a feature including but not limited to: a report of the statistically significant fraction of detection sites within the feature whose sensed state fall within a set of constraints, the average, mean, standard deviation, etc. of the values of conductivity or electrochemical current that were sensed at all the sites within a feature, and the like. The feature would be represented as the smallest set of detection sites whose state would be reported by such a statistically significant description.

The hybridization reactions involving biopolymers result in the ability to close an electrical circuit between the conductive layer edges and the conductive element in each of the detection sites in which a hybridization reaction occurs. Accordingly, a current flow above a particular threshold detected at a particular detection site indicates that a hybridization reaction has occurred at such site. Based on the knowledge of the oligonucleotides at each feature, information about the polynucleotide may be ascertained such as the presence and amount thereof by direct reporting of the fraction and nature of conductivity modification at the sites, the composition thereof and so forth.

In addition to the above-mentioned features, the device may also comprise identification codes, which may be either visual or electronic, to provide for interrogation of features of the device.

The results from the analysis involving exposing the substrate to the sample may optionally be processed. In this regard the results obtained from the aforementioned method may be processed by, for example, computer aided data analysis. In addition, the results may be forwarded to a remote location. By the term “remote location” is meant a location that is physically different than that at which the results are obtained. Accordingly, the results may be sent to a different room, a different building, a different part of city, a different city, and so forth. Usually, the remote location is at least about one mile, usually, at least ten miles, more usually about a hundred miles, or more from the location at which the results are obtained. The method may further comprise transmitting data representing the results. The data may be transmitted by standard means such as, e.g., facsimile, mail, overnight delivery, e-mail, voice mail, and the like.

The devices of the present invention may be provided as part of a kit useful for conveniently performing methods in accordance with the present invention. To enhance the versatility of the subject invention, the device can be provided in packaged combination with reagents for conducting the present methods such as sample pretreatment reagents, binding reagents, and the like. The reagents may each be in separate containers or various reagents can be combined in one or more containers depending on the cross-reactivity and stability of the reagents. The reagents may be, for example, those reagents used in current fluorescence-based DNA micro-arrays and in accord with current convention.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A device for sensing a targeted chemical reaction, said device comprising:
(a) a conductive layer having a plurality of openings therethrough, said conductive layer being at equipotential, and
(b) a plurality of separate conductive elements, each of said conductive elements being exposed within a respective opening in said conductive layer and separated therefrom by an insulating material to provide an exposed surface of said insulating material thereby forming a plurality of detection sites, said detection sites or groups thereof being electrically isolated from one another and being independently electrically addressable and readable.

2. A device according to claim 1 wherein said conductive layer is a metal layer.

3. A device according to claim 1 wherein said conductive elements are metal elements.

4. A device according to claim 1 wherein the exposed surface of the insulating layer has attached to it a reactant.

5. A device according to claim 1 wherein the surface of the conductive layer has attached to it a reactant.

6. A device according to claim 4 wherein said reactant is selected from the group consisting of biopolymers, electrochemically responsive compounds, and photochemically responsive compounds.

7. A device according to claim 4 wherein said reactant is a biopolymer selected from the group consisting of nucleotides, proteins, or protein receptors.

8. A device according to claim 5 wherein said reactant is selected from the group consisting of biopolymers, electrochemically responsive compounds, and photochemically responsive compounds.

9. A device according to claim 5 wherein said reactant is a biopolymer selected from the group consisting of nucleotides, proteins, or protein receptors.

10. A device according to claim 1 wherein the conductive layers are electrically addressable by virtue of an electronic switching device.

11. A device according to claim 10 wherein the switching devices are transistors.

12. A device according to claim 11 wherein said transistors are field effect transistors or bipolar transistors.

13. A device for sensing a targeted chemical reaction, said device comprising:
(a) a conductive layer having a plurality of openings therethrough, said conductive layer being at equipotential, and
(b) a plurality of separate conductive elements, each of said conductive elements being exposed within a respective opening in said conductive layer and separated therefrom by an insulating material to provide an exposed surface of said insulating material, thereby forming a plurality of detection sites, said detection sites or groups thereof being electrically isolated from one another and being independently electrically addressable and readable and being adapted to connect sequentially, individually or in groups, to a predetermined sense circuit or predetermined sets thereof wherein said sense circuit is adapted to render for each detection site a determination of the electrical activity adjacent a conductive element of said detection site.

14. A device according to claim 13 wherein said electrical activity is resistivity between said conductive layer and said conductive element of each of said detection sites.

15. A device according to claim 13 wherein said electrical activity is current produced at said conductive element of each of said detection sites.

16. A device according to claim 13 wherein each of said detection sites has a reactant attached thereto wherein a reactant at one detection site may be the same as or different from a reactant at another detection site and said determination of electrical activity determines a chemical or biological state of said detection site.

17. A device according to claim 13 wherein a group of detection sites is adapted to connect sequentially to predetermined sets of sense circuits and said electrical activity is resistivity between said conductive layer and said conductive element of each of said detection sites.

18. A device according to claim 13 wherein a group of detection sites is adapted to connect sequentially in a predetermined manner to a single sense circuit and said electrical activity is resistivity between said conductive layer and said conductive element of each of said detection sites.

19. A device according to claim 13 wherein each of said detection sites is adapted to connect sequentially to a predetermined sense circuit and said electrical activity is resistivity between said conductive layer and said conductive element of each of said detection sites.

20. A device according to claim 13 wherein each of said detection sites is adapted to connect sequentially to a predetermined sense circuit and said electrical activity is current produced at said conductive element.

21. A device according to claim 13 wherein a group of detection sites is adapted to connect sequentially to predetermined sets of sense circuits and said electrical activity is current produced at each conductive element respectively.

22. A device according to claim 13 wherein a group of detection sites is adapted to connect sequentially in a predetermined manner to a single sense circuit and said electrical activity is current produced at each conductive element respectively.

23. A device according to claim 13 wherein each sense circuit has associated circuitry to assemble a statistical description for a group of said detection sites.

24. A device according to claim 13 wherein a group of sense circuits has associated circuitry to assemble a statistical description for a group of said detection sites.

25. A device for sensing a targeted chemical reaction, said device comprising:
(a) a conductive layer having a plurality of openings therethrough, said conductive layer being at equipotential, and
(b) a plurality of separate conductive elements, each of said conductive elements being exposed within a respective opening in said conductive layer and separated therefrom by an insulating material to provide an exposed surface of said insulating material, thereby forming a plurality of detection sites, said detection sites or groups thereof being electrically isolated from one another and being independently electrically addressable and readable and being adapted to connect sequentially, individually or in groups, to a predetermined sense circuit or predetermined sets thereof wherein said sense circuit is adapted to render for each detection site a determination of the electrical activity adjacent a conductive element of said detection site and wherein each sense circuit, or a group thereof, has associated circuitry to assemble a statistical description for a group of said detection sites.

26. A device according to claim 25 wherein said electrical activity is resistivity between said conductive layer and said conductive element of each of said detection sites.

27. A device according to claim 25 wherein said electrical activity is current produced at said conductive element of each of said detection sites.

28. A device according to claim 25 wherein each of said detection sites has a reactant attached thereto wherein a reactant at one detection site may be the same as or different from a reactant at another detection site and said determination of electrical activity determines a chemical or biological state of said detection site.

29. A device according to claim 25 wherein a group of detection sites is adapted to connect sequentially to predetermined sets of sense circuits and said electrical activity is resistivity between said conductive layer and said conductive element of each of said detection sites.

30. A device according to claim 25 wherein a group of detection sites is adapted to connect sequentially in a predetermined manner to a single sense circuit and said electrical activity is resistivity between said conductive layer and said conductive element of each of said detection sites.

31. A device according to claim 25 wherein each of said detection sites is adapted to connect sequentially to a predetermined sense circuit and said electrical activity is resistivity between said conductive layer and said conductive element of each of said detection sites.

32. A device according to claim 25 wherein each of said detection sites is adapted to connect sequentially to a predetermined sense circuit and said electrical activity is current produced at said conductive element.

33. A device according to claim 25 wherein a group of detection sites is adapted to connect sequentially to predetermined sets of sense circuits and said electrical activity is current produced at each conductive element respectively.

34. A device according to claim 25 wherein a group of detection sites is adapted to connect sequentially in a predetermined manner to a single sense circuit and said electrical activity is current produced at each conductive element respectively.

35. An array comprising a plurality of distinct features, each of said features comprising a plurality of devices according to claim 1.

36. An array according to claim 35 wherein at least some of said features differ by comprising different reactants.

37. An array according to claim 25 wherein said associated circuitry to assemble a statistical description for a group of said detection sites is present on said device.

38. An array according to claim 37 wherein said device comprises a single substrate.

39. A method for assessing the status of detection sites on a substrate, said method comprising:
(a) acquiring data electronically from multiple detection sites on a single substrate and
(b) assembling a statistical description for a group of detection sites by means of sense circuitry on said substrate.

40. A method according to claim 39 wherein each of said detection sites has a reactant attached thereto.

41. A method according to claim 40 wherein said reactant is selected from the group consisting of biopolymers, electrochemically responsive compounds, and photochemically responsive compounds.

42. A method according to claim 40 wherein said reactant is a biopolymer selected from the group consisting of nucleotides, proteins, or protein receptors.

43. A method according to claim 39 wherein said detection sites are part of a reaction device, which comprises an array of distinct features, each of said features comprising a plurality of detection sites.

44. A method according to claim 43 wherein at least some of said features differ by comprising different reactants.
